SEQUENCE LISTING (39 sequences)

SEQ ID NO: 1 - DNA, 167 bp, Gallus gallus
ctgcctccagtcctgaactttctctgctgggatgagcagagttcacgctcggctgaactcgtgactgtgtcaacagggggcatgaagatcagcaccaggcaaaggtgagcggagtgcacaagaatgaggcagagacctttgggacaaagagttccccacccgtgggg

SEQ ID NO: 2 - DNA, 176 bp, Gallus gallus
gggctccatcggcctcttcaggttattgctatgttcaactcgtgtgaccttcctccctgttttcaaggttctgataacaaacttctaggacagcctggtgacctttggctcagcttcctctttaaactccggtggcttttgcttcattgcctagttgttacccagcagtaaagatc

SEQ ID NO: 3 - DNA, 175 bp, Mus musculus
tgagctcatcaggttcctggtggagagctgggtgaaccgagttcgtttgcactgccttggcctgtgtgtggcttcaggaacaggtcatgctccaggatttaggacacaggttagctgataaggcccatggagccggtgatatgacccgcagggtcactccctctgcccaagtcta

SEQ ID NO: 4 - DNA, 280 bp, Mus musculus
agtccagtcagaaccttctggcttctgccatgagacagcctatgagctcatcaggttcctggtggagagctgggtgaaccgagttcgtttgcactgccttggcctgtgtgtggcttcaggaacaggtcatgctccaggatttaggacacaggttagctgataaggcccatggagccggtgatatgacccgcagggtcactccctctgcccaagtctagatacttgtttccctctttagactccagatcaaggactccagatctgtcatggactcagaaaa

SEQ ID NO: 5 - DNA, 321 bp, Mus musculus
agtccagtcagaaccttctggcttctgccatgagacagcctatgagctcatcaggttcctggtggagagctgggtgaaccgagttcgtttgcactgccttggcctgtgtgtggcttcaggaacaggtcatgctccaggatttaggacacaggttagctgataaggcccatggagccggtgatatgacccgcagggtcactccctctgcccaagtctagatacttgtttccctctttagactccagatcaaggactccagatctgtcatggactcagaaaaccccccagtaccaccccacccccgctgctccctcctatgat

SEQ ID NO: 6 - DNA, 328 bp, Mus musculus
gtggctgggttgggatgggatgggaaggcttgtgtctctctgattcctagtccagtcagaaccttctggcttctgccatgagacagcctatgagctcatcaggttcctggtggagagctgggtgaaccgagttcgtttgcactgccttggcctgtgtgtggcttcaggaacaggtcatgctccaggatttaggacacaggttagctgataaggcccatggagccggtgatatgacccgcagggtcactccctctgcccaagtctagatacttgtttccctctttagactccagatcaaggactccagatctgtcatggactcagaaaa

SEQ ID NO: 7 - DNA, 369 bp, Mus musculus
gtggctgggttgggatgggatgggaaggcttgtgtctctctgattcctagtccagtcagaaccttctggcttctgccatgagacagcctatgagctcatcaggttcctggtggagagctgggtgaaccgagttcgtttgcactgccttggcctgtgtgtggcttcaggaacaggtcatgctccaggatttaggacacaggttagctgataaggcccatggagccggtgatatgacccgcagggtcactccctctgcccaagtctagatacttgtttccctctttagactccagatcaaggactccagatctgtcatggactcagaaaaccccccagtaccaccccacccccgctgctccctcctatgat

SEQ ID NO: 8 - DNA, 174 bp, Homo sapiens
gcgcaaagtcaacacaagcctctccaccgtgtgtccatgtttatgtgtatgcgctgtgccccgtcatgccacctggacgcagggactccagtgacctctccttgcacaagcctctgctggtttgggaaagattggcatgacatcagccaagctctggccttgccttttttccct

SEQ ID NO: 9 - DNA, 800 bp, Homo sapiens
caggcgcaaagtcaacacaagcctctccaccgtgtgtccatgtttatgtgtatgcgctgtgccccgtcatgccacctggacgcagggactccagtgacctctccttgcacaagcctctgctggtttgggaaagattggcatgacatcagccaagctctggccttgccttttttccctcccggaacccggctggctcaagatctgagccgtggatctgcacccactttggggagttcctgcccttgggctagagtagaggccaagagtcaaagtgtggtgggggctgaggcagcaggatcgcttgagcccaggagttcaaggttagagttatgtttgcatcactgcattccagcctgggtaagagagcgagaccctgcctcaaaaaaaaaaaaaaaaaaaaaaaaagagtggtgggggtagggacagggagatgaggaaggccctacagtggagaaagcaccaggaccagaacccagccctcccttgtctgaatcttgctgcccacaggagcctggacagtggccagggaaggttcgaatgccacacaggtgagcttggcctctgctctgtaggcagtgggaagtgctgggagttgggcattcctgtgaggcgcatagtcaacattgtgagtagggctggatgtcgagctgtgagagggaaactagaagctggaacatctacaggaggcttttaagagaagcagagcggccaggtgcagtggctgacacctgtaatcccagcactttgggtggccgagggggtcagaacacttgaggtcaggagttcgagaccagggtgac

SEQ ID NO: 10 - DNA, 935 bp, Homo sapiens
cacggtaccacaggaggcaaaggaccacccatggaatccacccaggagggccaggacaggggactcaggggcctcagcctgcactcacctgctcagcagagctgagctcagggcataacgtcagcttcctgggcagaagagctgccaaaatcaaagctttgctagtcagaaaattccttgggaatgttgagcaaggccaccactgacatcatgtgcaaattcgcagacagcctctgcacctaaggctactcagacccacagccttggctcgatggggtggcagactctgtatgccaccagcacacccacccacagggcagaggggtcaggacatagaatcagacaggccccagggaccccagtgaagattataggcagccattccccactcaacagaggagaaggtcagagccaagtctgacattcccccatcccctctccataacacccatgcatctggcagtcagacaggcctaagctaaacctttcccccccagctacccaccagggtcatccccaagccaggtcagggccaatggaggttggggtggagaagacaggcttggccctatttcctgcccaactcagaaccttctggtttctgccacaggatgccttgcaagcttatcggggtcactgtgggcagctgggtgagctaagttcatctgtgctgccgtgacctctgtgcagatgcatcaagaacacagagtgctccggggttaggatgagggcagcgctgataaggttcatggaaccagtgacagagcacacagctgcccacagagtcactcccctgtgccccagcctggacacctcagctccctctcaaccccttcccgaggtgctagatgtatatgggaccagaaagccccctctgtgtcctcctgtgtgagagcccagctgcttaggtgtttgtgactctgggcctcgagggg

SEQ ID NO: 11 - DNA, 25 bp, Artificial sequence
  Forward PCR primer
ggggaccagtccagtcagaaccttc

SEQ ID NO: 12 - DNA, 27 bp, Artificial sequence
  Reverse PCR primer
ccgctcgagttttctgagtccatgaca

SEQ ID NO: 13 - DNA, 26 bp, Artificial sequence
  Forward PCR primer
ggggtaccgtggctgggttgggatgg

SEQ ID NO: 14 - DNA, 27 bp, Artificial sequence
  Reverse PCR primer
ccgctcgagatcataggagggagcagc

SEQ ID NO: 15 - DNA, 32 bp, artificial sequence
  forward PCR primer
ggaggaactcgacacgataccaacatagcaat

SEQ ID NO: 16 - DNA, 30 bp, artificial sequence
  reverse PCR primer
ctatgttggtatcgtgtcgagttcctccct

SEQ ID NO: 17 - DNA, 28 bp, artificial sequence
  forward PCR primer
gaattcgccaactgcagccaggctgtcc

SEQ ID NO: 18 - DNA, 30 bp, artificial sequence
  reverse PCR primer
cagcctggctgcagttggcgaattctcctc

SEQ ID NO: 19 - DNA, 33 bp, artificial sequence
  forward PCR primer
ccccacgcagccccaccgctcggctgaactcgt

SEQ ID NO: 20 - DNA, 34 bp, artificial sequence
  reverse PCR primer
gtggggctgcgtggggcagcagagaaagttcagg

SEQ ID NO: 21 - DNA, 27 bp, artificial sequence
  forward PCR primer
gaattcacagccatggtgaagatcagc

SEQ ID NO: 22 - DNA, 25 bp, artificial sequence
  reverse PCR primer
ccatggctgtgaattcagtcacgag

SEQ ID NO: 23 - DNA, 24 bp, artificial sequence
  forward PCR primer
gtttaaagctggcactgtcccaaa

SEQ ID NO: 24 - DNA, 25 bp, artificial sequence
  reverse PCR primer
ctttggcacagtgccagctttaaac

SEQ ID NO: 25 - DNA, 27 bp, Artificial sequence
  Probe
ttccgccataacgacgtcaaccatctt

SEQ ID NO: 26 - DNA, 19 bp, artificial sequence
  forward PCR primer
gcagggtgccaaaacacat

SEQ ID NO: 27 - DNA, 24 bp, artificial sequence
  reverse PCR primer
tcgatggatcagacttcttcaaca

SEQ ID NO: 28 - DNA, 24 bp, artificial sequence
  Probe
tggcgtgcccattgatcacaattt

SEQ ID NO: 29 - DNA, 22 bp, artificial sequence
  forward PCR primer
ggtcacgctcctggaagatagt

SEQ ID NO: 30 - DNA, 20 bp, artificial sequence
  reverse PCR primer
gggcactgtcaaggctgaga

SEQ ID NO: 31 - DNA, 20 bp, artificial sequence
  Forward PCR primer
cgggcagcaggtcgaggaga

SEQ ID NO: 32 - DNA, 22 bp, artificial sequence
  reverse PCR primer
caggaacgggcattttgtagca

SEQ ID NO: 33 - DNA, 27 bp, artificial sequence
  reverse PCR primer
aggccaggcgaattcaacgaactcggt

SEQ ID NO: 34 - DNA, 27 bp, artificial sequence
  forward PCR primer
cgagttcgttgaattcgccttggcctg

SEQ ID NO: 35 - DNA, 26 bp, artificial sequence
  forward PCR primer
gccacacacctgcagaggcagtgcaa

SEQ ID NO: 36 - DNA, 27 bp, artificial sequence
  forward PCR primer
gcactgcctctgcaggtgtgtggcttc

SEQ ID NO: 37 - DNA, 27 bp, artificial sequence
  forward PCR primer
aattcgcctctgcaggtgtgtggcttc

SEQ ID NO: 38 - DNA, 26 bp, artificial sequence
  reverse PCR primer
gccacacacctgcagaggcgaattca

SEQ ID NO: 39 - DNA, 240 bp, Homo sapiens
tctggcagtcagacaggcctaagctaaacctttcccccccagctacccaccagggtcatccccaagccaggtcagggccaatggaggttggggtggagaagacaggcttggccctatttcctgcccaactcagaaccttctggtttctgccacaggatgccttgcaagcttatcggggtcactgtgggcagctgggtgagctaagttcatctgtgctgccgtgacctctgtgcagatgca

(19) United States
(12) Patent Application Publication
(10) Pub. No.: US 2005/0118601 A1
(43) Pub. Date: Jun. 2, 2005

(54) Enhancer sequence of the 5-aminolevulinic acid synthase gene

(76) Inventors: Urs Meyer, Oberwil (CH); David John Fraser, Binningen (CH); Michel R. Kaufmann, Basel (CH); Michael Podvinec, Basel (CH); Adrian Zumsteg, Basel (CH)

Correspondence Address: JOYCE VON NATZMER, 4615 NORTH PARK AVENUE, SUITE 919, CHEVY CHASE, MD, 20815, US

(21) Appl. No.: 10/510,678

(22) PCT Filed: Apr. 4, 2003
(86) PCT No.: PCT/IB03/01414

(30) Foreign Application Priority Data
Apr. 9, 2002 (IB) PCTIB0201258

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

Nucleic acid sequences mediating chemical compound induced 5-aminolevulinate synthase gene (ALAS1) expression are disclosed. Said sequences comprise at least a DR-4 binding site. Furthermore, in vitro methods for testing chemical compounds for modulation of heme and/or P 450 cytochromes synthesis are described.

### TECHNICAL FIELD

The present invention relates to a transcriptional enhancer of the 5-aminolevulinic synthase acid gene (ALAS1) and to a method for testing chemical compounds as inducers of heme and/or P450 synthesis.

### BACKGROUND ART

Humans are exposed to many foreign compounds (xenobiotics) in their diet, in their environment, and as clinically prescribed drugs (e.g., rifampicin and phenobarbital). In response to these exposures mammals have evolved mechanisms to induce proteins involved in xenobiotic detoxification. Metabolism by Phase I enzymes, particularly the heme containing monooxygenases cytochromes P450 is frequently the first line of defense against such xenobiotics. The activity of these detoxification enzymes leads to limited in vivo half lives of therapeutical drugs and consequently to a limited duration of the therapeutic effect.

Induction of drug-metabolizing enzymes by drugs and chemicals includes the transformation of drugs to inactive, active or toxic metabolites and has important clinical consequences. These consequences include drug-drug interactions and the precipitation of certain diseases such as the hepatic porphyrias. Induction of 5-aminolevulinic acid synthase by drugs, chemicals, hormones and nutrients is a hallmark of the acute attacks of hepatic porphyria, a rare inherited metabolic disease characterized by acute attacks of neuropsychiatric symptoms.

During the development of new therapeutial drugs it is therefore desirable to screen said new active compounds for their ability to induce detoxification enzymes.

The prior art describes test methods allowing a measurement of xenobiotic induction of degradation enzymes. International patent application WO 99/61622 describes a system for screening potential new drugs for susceptibility to metabolic degradation. Said method is based on a transcriptional enhancer of the human gene P450CYP3A4. Said enhancer is responsible for the transcriptional induction of the CYP3A4 gene by xenobiotic inducers including therapeutic drugs.

WO 99/48915 discloses a method of screening test compounds for their ability to induce CYP3A4 gene expression. The described method is based on the isolation of an orphan nuclear receptor designated human pregnane X receptor (hPXR) that binds e.g. to a rifampicin/dexamethasone response element in the CYP3A4 gene regulatory region. The binding of said hPXR receptor modulates transcription of the CYP3A4 gene. The CYP3A4 enzyme is just one of 55 human CYP enzymes of which many are inducible via similar or different enhancer regions.

Although there exist already test systems allowing an evaluation of the induction of degradation enzymes by xenobiotics such as e.g. new therapeutical drugs, there is still a need for alternative means and methods allowing an easy and inexpensive testing of new therapeutical drugs for their capacity to induce any drug-metabolizing enzyme.

### DISCLOSURE OF THE INVENTION

Hence, it is a general object of the invention to provide an isolated nucleic acid sequence which comprises at least a DR-4 nuclear receptor binding site and wherein said nucleic acid sequence functions as transcriptional enhancer of the 5-aminolevulinic acid synthase gene. Activation of said nucleic acid sequence is a marker for the induction of any cytochrome P450 gene and not just of CYP3A4.

In a preferred embodiment said nucleic acid sequence further comprises a nuclear factor 1 binding site (NF-1) and/or a DR-5 nuclear receptor binding site or has the sequence set forth in Seq. Id. No. 1.

The nucleic acid sequence of the present invention which encompasses a nuclear factor 1 binding site preferably comprises a sequence selected from the group consisting of Seq. Id. No. 2 to 7.

In a further preferred embodiment said nucleic acid sequences mediate chemical compound induced transcriptional activation. Said chemical compound is preferably a candidate compound for therapeutical use or a therapeutical drug.

Another object of the present invention is a genetic construct comprising a nucleic acid sequence of the present invention which is operably linked to a nucleic acid encoding a reporter molecule. Said reporter molecule has preferably an enzymatic activity, more preferably said reporter molecule activity can be detected by colorimetric methods, by radioactivity, fluorescence or chemiluminiscence.

Said reporter molecule is preferably selected from the group consisting of luciferase, beta-galactosidase, chloramphenicol acetyltransferase, alkaline phosphatase and green fluorescent protein.

A third object of the present invention is a method for testing compounds for modulation of heme and/or P450 cytochromes synthesis. Said method comprises the following steps: contacting suitable cells comprising a genetic construct of the present invention with a test compound and detecting enhanced/reduced expression and/or transcription of the nucleic acid sequence encoding the reporter gene. The detectable enhanced or repressed reporter gene expression and/or transcription is indicative of a compound that enhances or represses heme and/or P450 synthesis.

In a preferred embodiment said test compound is a candidate drug for therapeutical use or a therapeutical drug.

In a further preferred embodiment of the present method said enhanced expression of the nucleic acid sequence encoding the reporter gene is detected by colorimetry, fluorescence, radioactivity or chemiluminiscence.

In a particular preferred embodiment said enhanced transcription of the nucleic acid encoding the reporter gene is detected by quantitative PCR.

Preferred cells, for the use in a method of the present invention are Leghorn Male Hepatoma (LMH) cells, other hepatoma cells, monkey kindney cells (CV-1; COS-1) or human kidney cells.

In a further aspect the present invention relates to the use of a nucleic acid of the present invention or a fragment thereof for the testing of chemical compounds as modulators of heme and/or P450 synthesis.

The present invention relates furthermore to the use of a genetic construct of the present invention for the testing of chemical compounds as modulators of heme and/or P450 cytochromes synthesis.

### BRIEF DESCRIPTION OF THE DRAWINGS

The invention will be better understood and objects other than those set forth above will become apparent when consideration is given to the following detailed description thereof. Such description makes reference to the annexed drawings, wherein:

FIG. 1A shows the isolation of 176 bp and 167 bp drug-responsive enhancer sequences within the first 15 kb upstream of the chicken ALAS1 transcription start site by restriction endonuclease digestion and sub-cloning. Fragments were cloned into the pLucMCS luciferase reporter vector containing an SV-40 promoter;

FIG. 1B shows the DNA sequences of the 176 and 167 bp enhancers. Numbering refers to sequence positions relative to the transcriptional start site of the chicken ALAS1 gene. Solid lines identify DR4 and DR5 NR binding sites. Shaded boxes-contain individual half sites. A hatched line marks the NF1 binding site;

FIG. 1C shows reporter gene assays of the fragments. The constructs were transfected together with a transfection-control construct expressing β-galactosidase into LMH cells. Cells were then treated with 600 μM PB for 16 h and luciferase assays were performed on the cell extracts. Relative luciferase levels are standardized against cells transfected with vector containing no insert and expressed as fold induction. Experiments were repeated at least three times and data from a representative experiment tested in triplicate are shown here. Error bars represent standard deviations;

FIG. 2 shows a comparison of ADRES and mRNA activation by different drugs. Relative luciferase levels are standardized against cells transfected with vector containing no insert and expressed in fold induction. Experiments were repeated at least three times and data, from representative experiments tested in triplicate are shown here. Error bars represent standard deviations;

FIG. 3A shows site-directed mutagenesis of the DR4 and DR5 sites within the 176 bp ADRES element. Mutations in the DR4 and DR5 halfsites of the 176 bp sequences were introduced via PCR-based site-directed mutagenesis. Mutations are labeled in the left column and depicted with crosses in the scheme. Relative luciferase levels are standardized against cells transfected with vector containing no insert (control set to 1.0) and expressed as percentages of the 176 ADRES. Experiments were repeated at least three times and data from representative experiments tested in triplicate are shown here. Error bars represent standard deviations;

FIG. 3B shows site-directed mutagenesis of the DR4 sites within the 167 bp ADRES element. Mutations in the DR4 halfsites of the 167 bp sequence were introduced via PCR-based site-directed mutagenesis. Mutations are labeled in the left column and depicted with crosses in the scheme. The exprimental procedure was the same as described in FIG. 3A;

FIG. 4A shows a gel-mobility shift assay demonstrating that CXR binds the 176 bp ADRES element. Radiolabelled ADRES wild type (lanes 1-5) and mutant. (lanes 6-7) sequences were incubated With in vitro transcribed/translated CXR (lanes 3-7), chicken RXR (lanes 2 and 4-7) and anti-RXR antibody (Lane 5 and 7), as indicated. Arrows depict the unbound probe, the shifted CXR-RXR-probe complex, and the supershifted CXR-RXR-probe-anti-RXR antibody complex;

FIG. 4B shows gel-mobility shift assay demonstrating that CXR binds the 167 bp ADRES element. Radiolabelled ADRES wild type (lanes 1-5) and mutant (lanes 6-7) sequences were incubated with in vitro transcribed/translated CXR (lanes 3-7), chicken RXR (lanes 2 and 4-7) and anti-RXR antibody (lane 5 and 7), as indicated. Arrows depict the unbound probe, the shifted CXR-RXR-probe complex, and the supershifted CXR-RXR-probe-anti-RXR antibody complex;

FIG. 5A shows transactivation of the 176 bp ADRES element by CXR. Cos-1 cells were transfected with constructs containing 4 repeats of the wild type, DR4-1, DR5 and DR4-1/DR5 mutants cloned into the pBLCAT5 vector containing a thymidine kinase minimal promoter. The chicken CXR coding region cloned into the pSG5 expression vector was cotransfected along with a vector expressing pSV β-galactosidase as control. Cells were then treated for 16 h with either drugs or vehicle control and extracts were analyzed for CAT expression normalized against β-galactosidase levels as described in Materials and Methods. Experiments were repeated at least three times and data from representative experiments tested in triplicate are shown here. All constructs were verified by sequencing and error bars represent standard deviations;

FIG. 5B shows transactivation of the 167 bp ADRES element by CXR. Cos-1 cells were transfected with constructs containing a single copy of the wild type, DR4-2, DR4-3 and DR4-2/DR4-3 mutants cloned into the pBLCAT5 vector containing a thymidine kinase minimal promoter. The experimental procedure was the same as described under FIG. 5B;

FIG. 6 shows induction of expanded mouse core fragments in luciferase reporter gene assay in LMH cells. Inducer Metyrapone 500 μM;

FIG. 7 shows induction of the mouse 369 bp DRES by different drugs;

FIG. 8 shows the 369 bp mouse DRES sequence and discovered putative nuclear receptor binding sites;

FIG. 9 shows mutations introduced in the DR4-1 and DR4-2 sites of the 369 bp DRES sequence. The halfsites were mutated individually and both together. Mutated base pairs are underlined and in italics;

FIG. 10 shows induction of DR4 mutant constructs of mouse 369 DRES in luciferase reporter gene assay in LMH cells;

FIG. 11 shows drug-induction of different fragments of the human ALAS1 gene. Fragments were cloned into the pGL3 luciferase reporter vector (Promega Corp) and tested for inducibility by the prototypical hALAS1 inducers phenobarbital (PE) and propylisopropylacetamid (PIA). Four hours after transfection of LMH cells with the constructs, cells were exposed to the drugs for 24 hours, after which luciferase activity was assayed. Results were normalized for transfection efficiency by assaying for activity of co-transfected β-galactosidase. Data shown is one representative experiment;

FIG. 12 shows that the effect of drugs on the hA795 element depends on the presence of a DR-4 motif. Within the sequence of the hA795 fragment, a putative DR-4 type nuclear receptor response element was found by computer analysis. Site-directed mutagenesis of this element abolished inducibility of this fragment in reporter gene assays in LMH cells. Experiments were performed as described under FIG. 8;

FIG. 13 shows that a core sequence spanning the DR-4 element is sufficient to mediate drug induction in LMH cells. From the hA795 fragment, the hA174 fragment was derived. It is 174 bp in length and within its sequence, the DR-4 response element is contained. Direct repeats of the wildtype hA174 or a mutant, where the DR-4 was destroyed were cloned into the pGL3 reporter vector and tested in LMH cells;

FIGS. 14A and B show that the ALA synthase drug responsive enhancer sequence is inducible by mouse PXR (14A) and mouse CAR (14B) in transactivation assays;

FIGS. 15A and B show the results of gel shift assays of 369 bp ADRES with PXR (15A) and CAR (15B);

FIG. 16 shows the hA174 bp core element derived from the hA795 fragment conferring drug-mediated transcriptional activation to a reporter gene in LMH cells;

FIG. 17 shows the result of a drug induction assay in LMH cells using sub-fragments of the hA8 drug responsive element;

FIG. 18A depicts the hA174 bp core element of the drug responsive element within the human ALAS1 gene;

FIG. 18B depicts the hA240 bp core element of the drug responsive element within the human ALAS1 gene;

FIGS. 19A and B depict the results of an electrophoretic mobility shift assay, assaying the ability of human PXR and human CAR to bind to the hA174 bp and hA240 bp elements.

### MODES FOR CARRYING OUT THE INVENTION

Heme is an essential component in oxygen transport and metabolism in living systems. In non-erythropoietic cells, the first and rate-limiting enzyme in the pathway, 5-aminolevulinic acid synthase (ALAS1), regulates its biosynthesis. Under normal physiological conditions, free heme levels are low and tightly regulated, as toxicity can occur with increased cellular concentrations of unincorporated heme. Following administration of drugs such as phenobarbital (PB) or other prototypical CYP inducers, heme concentrations are elevated in the liver to accommodate the increased levels of heme dependent enzymes. This is achieved by induction of ALAS1 and assures an adequate and apparently coordinated supply of heme for the generation of functional cytochrome holoproteins such as e.g. cytochromes P450 (CYP).

In the scope of the present invention the inventors have identified and characterised nucleic acid elements in the 5′ flanking region of the gene encoding ALAS1 which functions as an enhancer for ALAS1 gene transcription. Said identified nucleic acid element is responsible for chemical compound induced ALAS1 gene transcription.

Said nucleic acid elements comprise at least a DR-4 nuclear receptor binding site. The term “DR-4 nuclear receptor binding site” as used herein refers to a direct repeat-4 hexamer repeat. Such a binding site is characterised by hexamer half sites arranged as direct repeats with a 4 nucleotide separation between half-sites. The half-site has the following canonical sequence AG(T/G)TCA. The term as used herein comprises as well functional equivalents of the canonical sequence i.e. half-site sequence variants which are still able to function as binding sites for nuclear receptors such as e.g. CAR (constitutive androstane receptor)/RXR (retinoid×receptor) heterodimers.

In a preferred embodiment said ALAS1 gene enhancer further comprises a NF-1 binding site. The term “NF-1 binding site” as used herein refers to a DNA element which serves as binding site for members of the nuclear factor-1 family of transcription factors, and said term encompasses functional equivalents thereof i.e. sequence variants which are still able to function as binding sites for members of the nuclear factor 1 (NF1) family of transcription factors. The NF-1 binding site has the following consensus sequence: TGGC(N₄)GCCA (N=any nucleotide). For a man skilled in the art it is clear that the sequences of the present invention can comprise more than one copy of the above identified binding sites.

In the scope of the present invention the following sequences conferring chemical compound induced ALAS1 gene transcription were characterised: Seq. Id. No. 1 (chicken), Seq. Id. No. 2 (chicken), Seq. Id. No. 3 to 7 (mus musculus), Seq. Id. No. 8 to 10 and Seq. Id. No. 39 (homo sapiens). The nucleic acid sequences set forth in Seq. Id. No. 8 to 10 and 39 have been part of a databank before the filing date of this application but these sequences have not been characterised and their function/activity has been unknown.

It has to be understood that the term nucleic acid sequence as used herein encompasses fragments, variants or derivatives of the sequences 1 to 10 of the present invention. Based on the disclosed enhancer sequences of ALAS1 and well known molecular biological methods as e.g. described in Sambrook et al., Molecular Cloning: A Laboratory Manual, New York: Cold Spring Harbor Laboratory, 2001) a man skilled in the art is able to isolate further sequences conferring chemical compound induced ALAS1 gene transcription/expression.

The construction of a genetic construct of the present invention can be done using standard molecular biology techniques as described e.g. in Sambrook et al., Molecular Cloning: A Laboratory Manual, New York: Cold Spring Harbor Laboratory, 2001).

The cell used in a test method can be any suitable cell allowing the performance of chemical compound induced assays. A particularly preferred cell line for the use in a method of the present invention is the Leghorn male hepatoma (LMH) cell line. The genetic construct can be introduced in the cells by well known transfection methods such as e.g. chemical transfection, elektrotransfection or viral transfection. Said host cell can express the genetic construct of the invention from a genomic locus or from an expression vector. Typically, an expression vector comprises the regulatory sequences required to achieve transcription and expression in the host cell and it may contain necessary sequences required for plasmid replication in order to exist in an episomal state, or it may be designed for chromosomal integration. A suitable vector is e.g. pGL3LUCpro (Promega) which comprises the gene encoding luciferase.

The method for testing compounds for modulation of heme and/or P450 synthesis of the present invention comprises the following steps: suitable cells harboring a genetic construct of the present invention are contacted with a test compound and an enhanced or repressed reporter gene expression and/or transcription is detected. The detection method of the enhanced or repressed gene transcription/expression is depending on the used reporter gene and can be done at the transcriptional level using e.g. quantitative PCR or by detecting the reporter gene product. Preferred detection methods for the reporter gene product are calorimetric, fluorescence or chemiluminiscence assays such as e.g. luciferase assay, CAT assay or β-galactosidase assay.

The invention is now further described by means of examples:

Isolation and Characterization of a Drug Responsive Enhancer of the Chicken 5-aminolevulinic Acid Gene

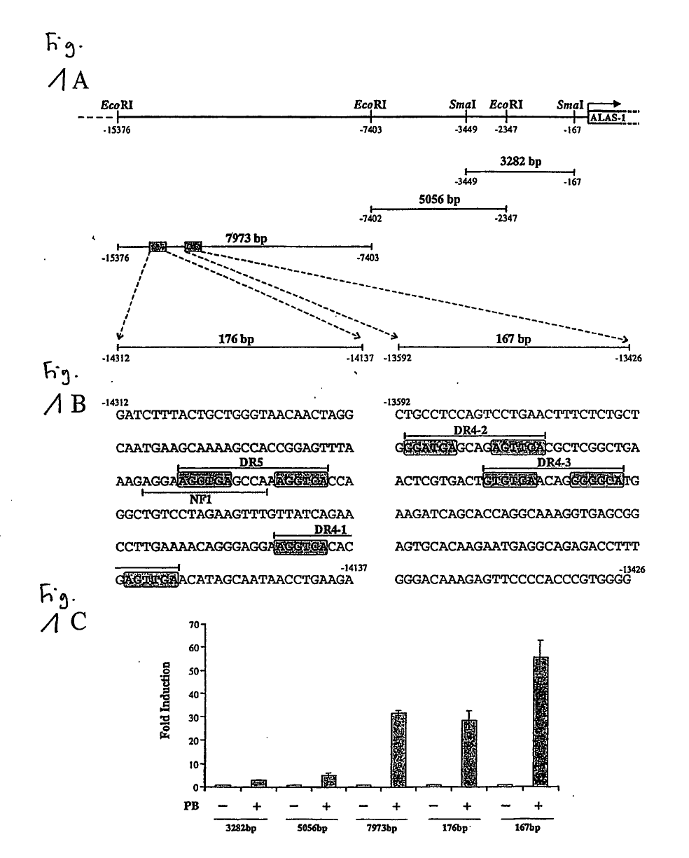

A cosmid clone containing an insert approximately 35 kb in length spanning the chicken ALAS1 gene and 15 kb of the 5′-flanking region, was isolated and its sequence analyzed. Three major subclones were generated from the region upstream of the transcriptional start site, including a 3282 bp SmaI fragment and 5056 bp and 7973 bp EcoRI segments (FIG. 1A). The SmaI clone extends from −167 bp to −3449 bp, whereas the EcoRI subfragments span the regions from −2347 bp to −7402 bp and −7403 bp to −15376 bp, respectively. These subfragments were cloned into the pLucMCS modified luciferase vector containing an SV40 promoter as described in Materials and Methods. Drug inducibility was measured in transiently transfected LMH cells treated with 600 μM PB and compared with control values. The results revealed the 7973 bp subfragment to be highly inducible with PB, displaying a 28-fold increase in transcriptional activation relative to control values. In comparison, the 5056 bp and 3282 bp subfragments exhibited virtually no transcriptional activation in response to drug treatment (FIG. 1C). The 7973 bp subfragment (−15376-7403) was chosen for further analysis and was divided into numerous subclones in the pLucMCS reporter vector resulting in the isolation of 176 bp Sau3AI-SmaI and 167 bp PvuII-HaeIII elements (FIGS. 1A and 1B). These sequences routinely exhibit 25-60 fold induction over control values in reporter gene assays when exposed to PB in LMH cells (FIG. 1C). All other portions of the 7973 bp fragment were also subcloned but displayed no drug response when tested in LMH cells. Because the 176 bp (Seq. Id. No. 2) and 167 bp (Seq. Id. No. 1) fragments retain high drug response regardless of orientation or distance from the promoter they are referred to as aminolevulinic acid synthase drug responsive enhancer sequence (ADRES) elements.

Recent discoveries have implicated NRs in drug mediated enzyme induction (2, 3, 8). For this reason, we scanned the responsive elements for potential nuclear receptor response sites using a computer algorithm based on a weighted nucleotide distribution matrix compiled from published functional hexamer halfsites. Two potential binding sites for orphan NRs were identified in each ADRES element, having two direct repeats with 4 nucleotide (DR4) and 5 nucleotide (DR5) separations between halfsites in the 176 bp sequence and two direct repeats with 4 nucleotide (DR4) separations between halfsites in the 167 bp sequence (FIG. 1B). For clarity, the three DR4 binding sites are labeled according to their occurrence in the gene, with the furthest upstream from the transcription start site called DR4-1 and the closest to the start site DR4-3. The putative DR4-1 is defined by one perfect half-site (AGGTCA) and one imperfect half-site (AGTTGA) at −14186/−14181 and −14176/−14171 respectively, whereas the DR5 site is characterized by an imperfect upstream half-site (AGCTGA) and a perfect down-stream half-site (AGGTCA) at −14251/−14246 and −14240/−14235. In the 167 bp sequence, DR4-2 consists of one imperfect upstream half-site (GGATGA) and one perfect down-stream half-site (AGTTCA) at −13563/−13558 and −13553/−13548 and DR4-3 has two imperfect halfsites (GTGTCA and GGGGCA) at −13526/−13521 and −13516/−13511. It is interesting to note that the 176 bp ADRES also contains a putative binding site for nuclear factor 1 which overlaps the DR5, spanning bp −14255 to bp −14242, whereas the 167 bp ADRES does not.

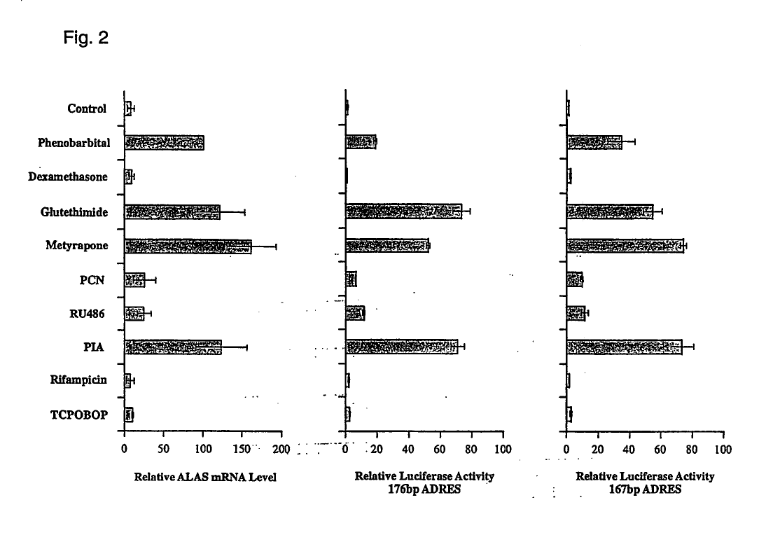

We next wanted to compare ADRES-mediated ALAS1 induction levels from reporter gene assays with stimulation of transcription in a physiological system. Therefore, ALAS1 mRNA levels were quantified in LMH cells cultured in serum-free medium and 16 h of exposure to a variety of chemical inducers and compared to the induction pattern observed with the same compounds in transient transfections of the ADRES (FIG. 2). The compounds examined include PB (600 μM) and the PB-like inducers PIA (250 μM), glutethimide (500 μM), and the potent mouse CYP 2B inducer 1,4-bis[2-(3,5-dichloropyridyl-oxy)]benzene (TCPOBOP) (10 μM). In addition, the common CYP3A inducers dexamethasone (50 μM), metyrapone (400 μM), and 10 μM mifepristone (RU-486) were employed for comparison. We were also interested in the effects of 10 AM 5-pregnen-3β-ol-20-one-16α-carbonitrile (PCN) and rifampicin (100 μM) due to their species-specific effects on PXR activation and CYP3A induction. Messenger RNA was reverse transcribed and levels of ALAS1 cDNA were quantified using the Taqman real-time PCR quantification system as described in Materials and Methods. PB was a strong inducer of ALAS1 in LMH cells, increasing RNA levels an average of 16 fold relative to basal transcript levels (FIG. 2). This value was chosen to represent 100% induction, against which all other values are compared. The general inducers PIA and glutethimide, as well as the 3A-specific inducer metyrapone exhibited the strongest effects upon the ADRES elements, stimulating transcription in excess of levels obtained from PB treatment. In comparison, dexamethasone, PCN, RU-486, and rifampicin had minor or no effects on either mRNA levels or ADRES activation. Moreover, the mouse-specific compound TCPOBOP elicited no response in either mRNA transcription or stimulation of the ADRES in reporter assays. When comparing the induction profiles of the two ADRES elements to each other, very few differences are in evidence. The 167 bp (Seq. Id. No. 1) responds to PB with twice the activation when compared to the 176 bp element (Seq. Id. No. 2). Also, the 176 bp has slightly more affinity for glutethimide than metyrapone, whereas the 167 bp element exhibits a stronger response to metyrapone than glutethimide. These experiments indicate a high degree of similarity in the relative activation of the ADRES elements in reporter gene assays to each other and to mRNA transcript levels from chemically induced LMH cells.

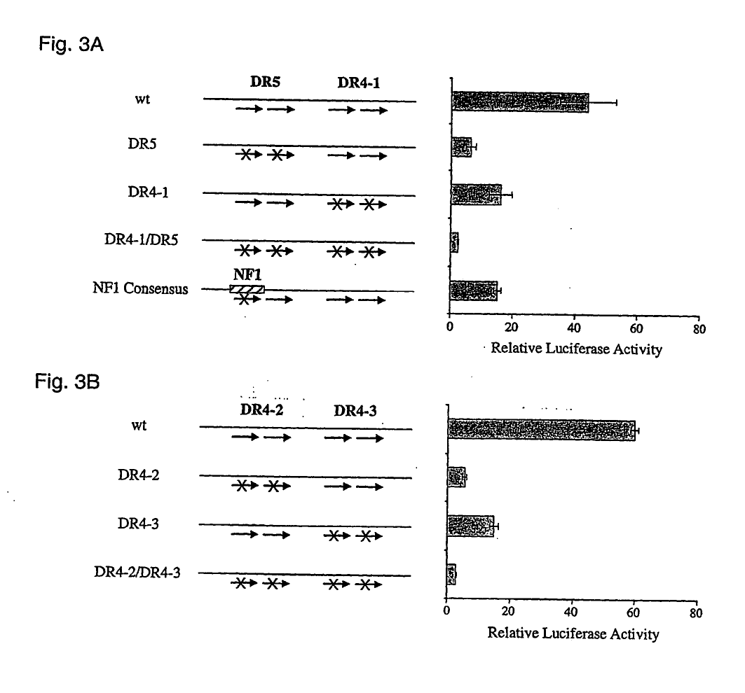

Site-specific mutagenesis was used to examine the roles of specific nucleotides within the putative DR5 and DR4 recognition sequences in conferring drug response to the ADRES elements (FIG. 3). Mutant constructs of the DR4 and DR5 core recognition sites destroying the putative NR binding sites were generated as described in Materials and Methods. Briefly, primers were used in conjunction with PCR to convert the 5′ and 3′ half-sites of the DR5 to EcoRI and PstI restriction endonuclease sites, respectively. Similarly, the DR4-3 half-sites were converted to EcoRI and NcoI restriction endonuclease sites. Data from a nucleotide distribution matrix for halfsites developed by M. Podvinec in this laboratory was applied to ascertain that the mutated halfsites least resemble functional halfsites. DR4-1 halfsites were obliterated by converting AGGTCA and AGTTGA halfsites to unconserved ACTCGA and ATACCA bases, respectively. Similarly, DR4-2 halfsites were both converted from GGATGA and AGTTCA nucleotides to CCCCAC bases. Primers were used to generate constructs mutated at each individual and both NR binding sites within both of the ADRES elements as shown in FIG. 3.

The modified enhancers were examined for response to 600 μM PB in luciferase reporter gene assays and the results are presented in FIG. 3. These findings indicate that both the DR5 and DR4 recognition sites in the 176 bp ADRES and both DR4 recognition sites in the 167 bp ADRES element are essential to elicit full drug response. Mutation of the DR5 reduced activity of the 176 bp ADRES element by over 85% from 44 fold to 6.4 fold activation by PB, whereas changes in the DR4-1 limited activation by over 60% from 44 fold to 16 fold stimulation (FIG. 3A). As depicted in FIG. 3B, both DR4-2 and DR4-3 sites in the 167 bp ADRES element were found to be required for full activation by PB. Alteration of the DR4-2 site resulted in the reduction of PB response by over 90% from 60 fold to 5.4 fold. Mutations in the DR4-3 site caused PB response to be diminished 75% from 60 to 15 fold induction. These studies demonstrate an essential contribution of the sequences within the putative DR4 and DR5 NR binding sites to PB activation of the ADRES elements.

Because the DR5 overlaps a putative binding site of NF1, a transcription factor that has been implicated in modifying drug induction, we tested the possibility that NF1 confers activation to the 176 bp ADRES element rather than NRs binding to the DR5 (5). A mutant construct converting the putative NF1 site to a consensus avian NF1 binding site, thus destroying the first half-site of the DR5, was generated and tested in luciferase assays. As seen in FIG. 3A, the NF1 consensus sequence does not increase the response of the 176 bp ADRES element to drugs. Rather, the induction is decreased by 66 percent from 44 to 15 fold induction, presumably due to the destruction of the DR5 NR binding site. In order to confirm these findings, chicken NF1-A was amplified from a cDNA library generated from LMH cells and cloned into pSG5 expression vector. Coding sequence fidelity was confirmed by sequencing and the construct was co-transfected both in induction experiments in LMH cells and transactivations in Cos-1 cells, resulting in no changes in induction or transactivation.

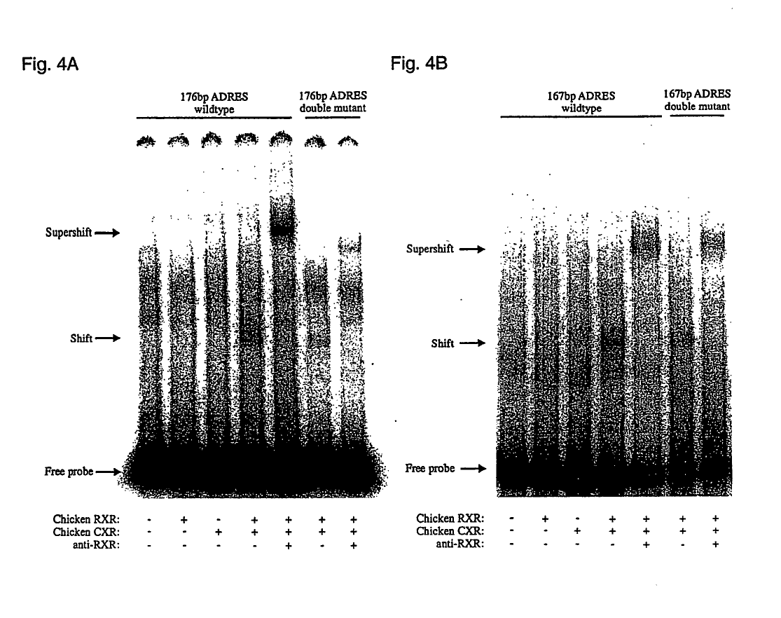

Recent findings have implicated a number of orphan NRs in drug induction of cytochromes P450 (for reviews, see (6, 10, 11). Our group has successfully cloned and expressed chicken CXR and has demonstrated CXR-RXR interactions with CYP enhancers in electrophoretic mobility shift assays (EMSAs) (2). As the DR4 and DR5 sites clearly contribute to the transcriptional activation exhibited by the ADRES elements, gel-mobility shift assays were used to determine whether CXR might bind the responsive enhancers (FIG. 4). Neither in vitro transcribed/translated chicken CXR nor chicken RXR alone bound to the ³²P-radiolabeled 176 bp ADRES (FIG. 4A, lanes 2 and 3) or to the 167 bp ADRES (FIG. 4B, lanes 2 and 3). In contrast, CXR/RXR heterodimers bind the drug responsive enhancers, and these complexes could be supershifted with anti-RXR antibodies (FIGS. 4A and 4B, lanes 4 and 5). Nuclear receptor binding to the 167 bp ADRES element was reduced when the double mutant DNA sequences were used, as demonstrated by the reduced band intensities of the shifted and supershifted components (FIG. 4B, lanes 6 and 7). Moreover, the binding of CXR/RXR heterodimers was virtually eliminated when both binding sites in the 176 bp ADRES elements were mutated (FIG. 4A, lanes 6 and 7). These findings demonstrate interactions of CXR/RXR heterodimers with the ADRES elements through the DR4 and DR5 NR binding sites.

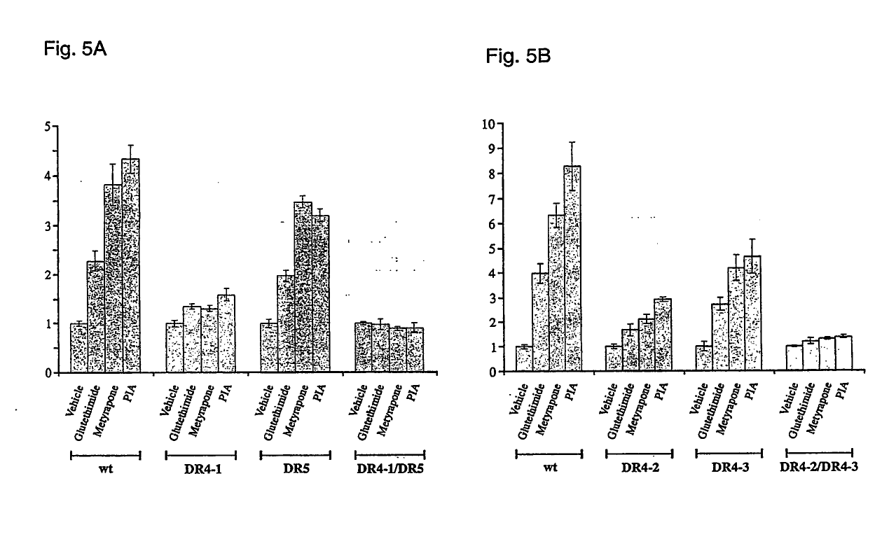

In order to confirm the role of CXR in the activation of the ADRES elements, transactivation experiments were done in Cos-1 monkey kidney cells. These cells express RXR but exhibit no induction response under normal conditions. Four copies of the wild type and mutated 176 bp element or a single copy of the mutated and wild type 167 bp element were cloned into the pBLCAT5 plasmid containing a tk minimal promoter as described in Materials and Methods. CAT vectors were used for transactivations rather than luciferase because CAT provided more stable expression and showed higher drug response. These constructs were cotransfected along with a pSG5 expression vector containing the coding sequence for CXR and a βgalactosidase expression construct to correct for variations in transfection efficiency. After 24 h incubation to allow for the expression of CXR, induction of the wild type and mutant sequences was tested with glutethimide, metyrapone and PIA, the three best inducers identified in FIG. 2. As shown in FIG. 5, both the 176 bp and 167 bp ADRES elements are transactivated by CXR. n the 176 bp element, the induction was reduced by 10-25% in the constructs carrying the mutant DR5 NR binding site. The mutations in the DR4-1 binding site reduced the induction by all drugs to less than 1.6 fold. Moreover, the alteration of both NR binding sites in the 176 bp element resulted in the complete elimination of drug response. The 167 bp element was found to respond better to drugs in transactivatons than the 176 bp element, thus a single copy was sufficient. The induction of the wild type sequence was strong for all three drugs, ranging from 4.0 to 8.3 fold over uninduced levels (FIG. 5B). The DR4-2 mutants exhibited lower induction after drug exposure, reduced by 58-66% when compared to wild type values. Similarly, the DR4-3 mutant sequences responded to drugs with diminished capacity, exhibiting 55-66% of the 167 bp activity. The double mutant 167 bp element did not respond to drugs, confirming the role of CXR in activating the ADRES elements via the DR4 and DR5 NR binding sites.

Material and Methods

Reagents

Dexamethasone, metyrapone (2-methyl-1,2-di-3-pyridyl-propadone), 5-pregnene-3β-ol-20-one-16α-carbonitrile (PCN) and rifampicin were purchased from Sigma chemical company. Propylisopropylacetamide (PIA) was a gift from Dr. Peter Sinclair (Veterans Affairs Hospital, White River Junction, Vt.). Glutethimide was purchased from Aldrich. Mifepristone (RU-486) was obtained from Roussel-UCLAF. 1,4-Bis[2-(3,5-dichloropyridyloxy)]benzene (TCPOBOP) was generously provided by U. Schmidt (Institute of Toxicology, Bayer, Wuppertal, Germany). Phenobarbital sodium salt (5-ethyl-5-phenyl-barbituric acid sodium salt) was purchased from Fluka. Tissue culture reagents, media, and sera were purchased from Life Technologies. All other reagents and supplies were obtained from standard sources.

Plasmids

The pGL3LUC luciferase reporter containing an SV40 promoter was purchased from Promega. The reporter plasmid was modified by the addition of the fragment spanning the SacI to the XhoI restriction endonuclease sites of the multiple cloning site of the pBluescript SK vector (Stratagene) to the pGL3LUC vector, thus greatly enhancing the cloning versatility of the new pLucMCS reporter. The pBLCAT5 chloramphenicol acetyl transferase reporter vector was described previously (1). Chicken CXR and RXR were cloned into the pSG5 expression vector (Stratagene) as previously reported (2). The pRSV β-galactosidase vector used for normalization of transfection experiments was kindly provided by Anastasia Kralli (Biozentrum, University of Basel, Basel, Switzerland).

Cosmid Isolation

A specific probe for the ALAS1 gene was generated via PCR using chicken embryo liver genomic DNA as template and forward primer 5′-CGG GCA GCA GGT CGA GGA GA-3′ (Seq. Id. No. 31) and reverse primer 5′-CAG GAA CGG GCA TTT TGT AGC A-3′(Seq. Id. No. 32). The probe was ³²P-radiolabeled using the random primer labeling kit (Roche Molecular Biochemicals) according to the manufacturer's instructions. A genomic cosmid library generated from adult male Leghorn chicken liver was purchased from Clontech Laboratories. The ALAS1 probe was used to identify an individual cosmid clone containing the ALAS1 gene and at least 15 kb of 5′-flanking region was isolated and confirmed by sequencing.

Construction of Vectors

The cosmid containing the ALAS1 gene and flanking region was digested with EcoRI restriction endonuclease and subfragments of the approximately 35 kb of new sequence were cloned into the EcoRI site of the pLucMCS vector. Eight fragments ranging in size from 10 kb to 900 bp in length were cloned. In addition, a 3282 bp SmaI fragment encoding the ALAS1 promoter region and proximal 5′-flanking region was cloned into pLucMCS. The drug-responsive 8 kb EcoRI region was then further subdivided using standard subcloning procedures and restriction endonucleases to isolate the Sau3AI-SmaI 176 bp element and the PvuII-HaeIII 167 bp element. Single copies of the 176 bp and 167 bp wild type and mutated elements were cloned into pBLCAT5 by excising a 222 bp fragment containing the desired sequences with BamHI and BglII restriction endonucleases and ligating them into BamHI-linearized pBLCAT5 vector. Multiple repeats of the 176 bp wild type and mutant elements were subcloned by inserting the 222 bp fragment 4 times in succession into the BamHI-linearized pBLCAT5 vector.

Cell Culture

Leghorn Male Hepatoma (LMH) cells were obtained from the American Type Culture Collection and cultivated in 10 cm dishes in Williams E medium supplemented with 10% FCS, 1% glutamine (2 mM) and 1% penicillin/streptomycin (50 IU/ml). Dishes coated with 0.1% gelatin were used for routine culture of LMH cells in order to facilitate proper seating of the cells onto the plastic plate surface. For transfections, cells were seeded onto 12-well Falcon 3043 dishes and expanded to 70-80% surface density. Cells were then maintained in serum-free Williams E media for 24 hours and transfected using the FuGENE 6 transfection reagent (Roche Molecular Biochemicals) according to the manufacturers protocol.

Analysis of Reporter Gene Expression

Cells were treated with drugs or vehicle for 16 h and harvested. For luciferase assays, lysis was performed with 200 μl Passive Lysis Buffer (Promega) per well and extracts were centrifuged for 1 minute to pellet cellular debris. Luciferase assays were performed on supernatants using the Luciferase Assay kit (Promega) and a Microlite TLX1 luminometer (Dynatech). Relative β-galactosidase activities were determined as described (4). For CAT assays, cells were lysed with 600 μl CAT lysis buffer per well and extracts were centrifuged for 1 minute to pellet cellular debris. Assays were performed using a CAT ELISA kit (Roche Molecular Biochemicals) according to the manufacturers protocol.

Site-Directed Mutagenesis

Mutations in the putative NR binding sites were introduced into the ADRES elements by PCR using standard overlap techniques. Briefly, subfragments were amplified with overlapping primers carrying the desired mutations and vector primers. These subfragments were then combined and used as template in a second PCR using vector primers to amplify the full-length mutated fragment, which was subsequently digested with appropriate enzymes and cloned into pLucMCS. The forward vector primer was the RV primer 3 and the reverse vector primer was the GL primer 2 within the pGL3 luciferase vector (Promega). All mutations are shown in bold. DR4-1 double mutation constructs were generated with 5′-GGA GGA ACT CGA CAC GAT ACC AAC ATA GCA AT-3′ forward (Seq. Id. No. 15) and 5′-CTA TGT TGG TAT CGT GTC GAG TTC CTC CCT G-3′ reverse (Seq. Id. No. 16) primers. DR5 double mutants were amplified with 5′-GAA TTC GCC AAC TGC AGC CAG GCT GTC C-3′ forward (Seq. Id. No. 17) and 5′-CAG CCT GGC TGC AGT TGG CGA ATT CTC CTC-3′ reverse (Seq. Id. No. 18) primers. DR4-2 double mutants were generated with 5′-CCC CAC GCA GCC CCA CCG CTC GGC TGA ACT CGT G-3′ forward (Seq. Id. No. 19) and 5′-GTG GGG CTG CGT GGG GCA GCA GAG AAA GTT CAG G-3′ reverse (Seq. Id. No. 20) primers. DR4-3 double mutants were amplified using a 5′-GAA TTC ACA GCC ATG GTG AAG ATC AGC-3′ forward (Seq. Id. No. 21) primer and a 5′-CCA TGG CTG TGA ATT CAG TCA CGA G-3′ reverse (Seq. Id. No. 22) primer. Avian NF1 consensus sequence was generated using 5′-GTT TAA AGC TGG CAC TGT CCC AAA-3′ (Seq. Id. No. 23) and 5′-CTT TGG CAC AGT GCC AGC TTT AAA C-3′ (Seq. Id. No. 24) forward and reverse primers (9). Following PCR overlap, the products were digested with BglII and either EcoRI or NotI restriction endonucleases and cloned into pLucMCS. All constructs were verified by sequencing.

Quantitative PCR

LMH cells were plated onto 12-well plates, expanded to 70-80% surface density and incubated in serum-free media for 24 h. Cells were then exposed to either drug or vehicle and RNA was isolated with Trizol reagent (Gibco BRL) according to the manufacturer protocol. One μg of total RNA was reverse transcribed with the Moloney murine leukemia virus reverse transcriptase kit (Roche Molecular Biochemicals). PCR was performed using the Taqman PCR core reagent kit (PE Applied Biosystems) and transcript levels quantitated with an ABI Prism 7700 sequence detection system (PE Applied Biosystems). Relative transcript levels were determined using the relative quantitation method measuring the ΔΔCt. The following primers and probes were used in these reactions. ALAS1: probe, 5′-TTC CGC CAT AAC GAC GTC AAC CAT CTT-3′(Seq. Id. No. 25); forward primer, 5′-GCA GGG TGC CAA AAC ACA T-3′ (Seq. Id. No. 26); reverse primer, 5′-TCG ATG GAT CAG ACT TCT TCA ACA-3′(Seq. Id. No. 27). Glyceraldehyde-3-phosphate dehydrogenase (GAPDH): probe, 5′-TGG CGT GCC CAT TGA TCA CAA TTT-3′ (Seq. Id. No. 28); forward primer, 5′-GGT CAC GCT CCT GGA AGA TAG T-3′ (Seq. Id. No. 29); reverse primer, 5′-GGG CAC TGT CAA GGC TGA GA-3′ (Seq. Id. No. 30). Transcript levels were measured in separate tubes and GAPDH values were used for normalization of ALAS1 values.

Gel Mobility-Shift Assays

Chicken CXR and RXR proteins were expressed using the TNT T7 Quick Coupled Translation System (Promega) according to the manufacturers protocol. Probes were labeled by Klenow reaction in the presence of radiolabeled [α-³²P]ATP and purified over a Biospin 6 chromatography column. A volume of labeled oligonucleotide corresponding to 100,000 cpm was used for each reaction in 10 mM Tris-HCL (8.0)/40 mM KCl/0.05% Nonidet P40′/6% glycerol (vol./vol.)/1 mM DTT containing 0.2 μg of poly(dI-dC) and 2.5 μl in-vitro synthesized proteins as described previously (2, 7). To test for supershifts, 0.5 μl monoclonal anti-mouse-RXR rabbit antibody (kindly provided by P. Chambon, Université Louis Pasteur, Illkirch, France) were added to the reaction mix. This antibody has been previously tested for interactions with chicken RXR in Western blots (data not shown). The reaction mix was incubated for 20 min at room temperature and electrophoresed on a 6% polyacrylamide gel in 0.5× Tris/borate/EDTA buffer followed by autoradiography.

Transactivations

Experiments to determine the contribution of the nuclear receptor CXR to the induction of ALAS-1 were tested in Cos-1 monkey kidney cells (generously provided by A. Kralli, Biozentrum, University of Basel, Basel, Switzerland) according to methods previously described (2). Briefly, cells were expanded for three days on 10 cm Falcon 3003 dishes in DMEM/F12 medium (Gibco BRL) without phenol red supplemented with 10% charcoal-stripped FBS. Cells were then plated onto 6-well dishes and expanded overnight to approximately 30% density. Cells were then rinsed with PBS and maintained for transfection in Optimem (Gibco BRL) without further additions. Transfection of 1 μg reporter plus 800 ng of pSV β-galactosidase construct and 50 ng of CXR expression vector was performed using 3 ml of LipofectAMINE (Invitrogen) per well, according to the manufacturer protocol. After 24 h incubation, cells were rinsed with PBS and DMEM/F12 containing 10% delipidated/charcoal-stripped FBS containing either drugs or vehicle control was added. After 16 hours induction, cells were rinsed with PBS and lysed in 600 μl CAT lysis buffer and assayed for CAT enzyme using the CAT-ELIZA kit (Roche Molecular Biochemicals). CAT levels were then normalized against β-galactosidase levels to compensate for variations in transfection efficiency.

Isolation and Characterisation of a Drug Responsive Enhancer of the mouse 5-aminolevulinic Acid Gene

Cloning of the 5′ Flanking Region of mALAS1

As only several hundred base pairs sequence information of mALAS1 were known, the strategy of isolating 5′ flanking region was “chromosomal walking” by southern blotting. For this purpose two bacterial artificial chromosome (BAC) clones termed 113 and 266 were used (BAC 113d22 and 266n18, mouse C57 B/6 from Genome Systems Inc., St. Louis, Mo., USA). By application of said method four different clones, spanning about 17 kb of flanking region were identified. Of these clones a 2.6 kb fragment was “inducible” in reporter gene assays.

The 2.6 kb HindIII fragment (−14.7 kb to −17.3 kb) was cloned as follows: 3μg 266 were digested with HindIII over night. A 0.7% gel was run for 6 hours at 90V and the 2.6 kb band was extracted and ligated to HindIII cut and dephosphorylated, gel-purified pBS bluescript (Stratagene, La Jolla, Calif., USA) and heat-shock transformed. Subcloning into pGL3LUCpro +MCS was done using EcoRI and KpnI, so that the fragment was in forward direction. In luciferase reporter assay using a LMH (Leghorn male hepatoma) cell system said construct showed drug inducibility.

Identification of Drug Responsive Enhancer Sequence (DRES) in the 2.6 kb HindIII Fragment

The inventors of the present invention have found that the sequence in the priority application PCT/IB02/01258 designated as 2.6 kb HindIII fragment has acutally a length of 2.8 kb in the genome. The cloning of the fragment in bacteria resulted in the elimination of 0.2 kb without functional consequences.

The 2.6 kb fragment was completely sequenced. The fragment has a length of 2604 bp. For easy orientation, numbering was given starting at 1 for the 5′ end of said fragment. The following fragments were amplified using the 2.6 kb fragment as template: 280 bp fragment (398 to 677) (Seq. Id. No. 4), 321 bp fragment (398 to 718) (Seq. Id. No. 5), 328 bp fragment (350 to 677) (Seq. Id. No. 6) and 369 bp fragment (350 to 718) (Seq. Id. No. 7. The following primer pairs were used:
280 bp fragment: mALAS-16.6FwdKpn (Seq. Id. No. 11) and mALAS-16.35rvXhoI (Seq. Id. No. 12).
328 bp fragment: mALAS-16.65fwdKpn (Seq. Id. No. 13) and mALAS-16.35rvXhoI (Seq. Id. No. 12).
321 bp fragment: mALAS-16.6fwdKpn (Seq. Id. No. 11) and mALAS-16.3rvXhoI (Seq. Id. No. 14).
369 bp fragment mALAS-16.65fwdKpn (Seq. Id. No. 13) and mALAS-16.3rvXhoI (Seq. Id. No. 14).

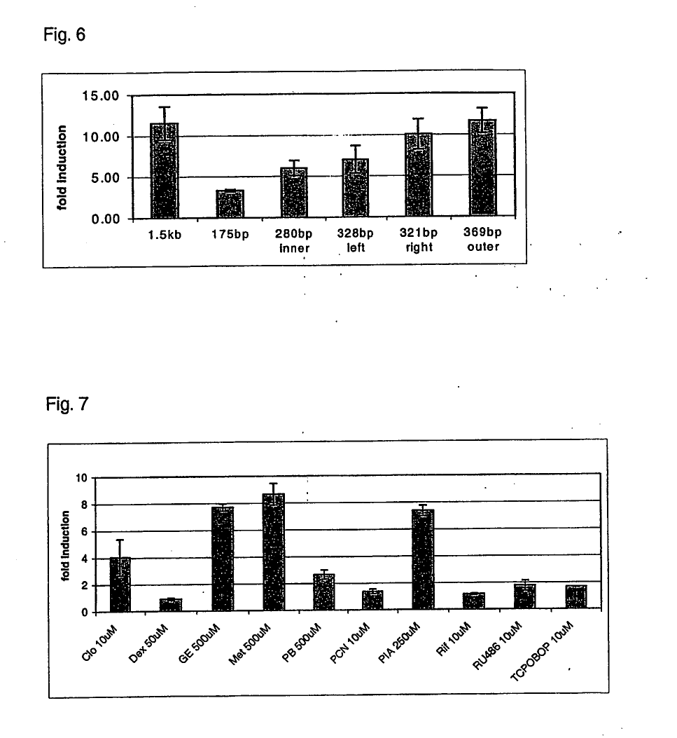

A 175 bp fragment (441 to 615) (Seq. Id. No. 3) was isolated by digesting the 2.6 kb fragment with SacI/XbaI. All fragments were subcloned in the pGL3LUCpro+MCS vector and the resulting constructs tested for inducibility in LMH reporter gene assay (FIG. 6). All five fragments showed enhanced induction with as inducer Metyrapone 500 μM. The 369 bp fragment was termed Drug responsive enhancer sequence DRES.

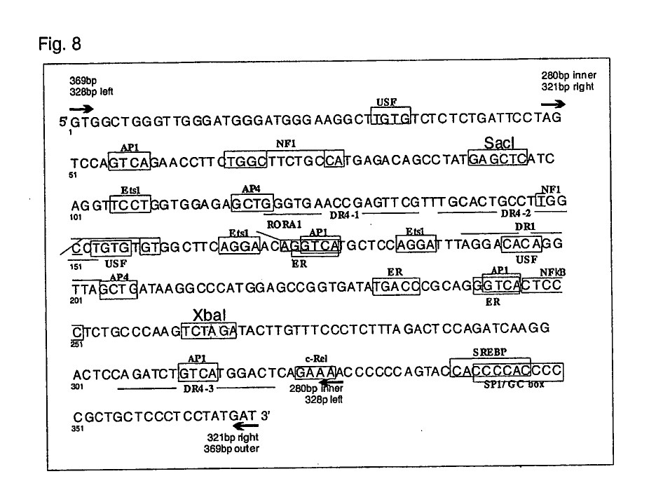

The 369 bp DRES sequence and discovered putative nuclear receptor binding sites are shown in FIG. 8. Putative nuclear receptor binding sites were discovered by MatInspector using a core similarity of 0.8 and matrix similarity of 0.85. Putative binding sites include: upstream stimulatory factor (USF), activator protein ¼ (AP1/AP4), nuclear factor 1 (NF-1), CAR/CXR/PXR (DR4), ets-1, estrogen receptor (ER), RAR-related orphan receptor alpha1 (RORA1), PPAR (DR1), nuclear factor κ B (NFκB), c-Rel, sterol response element binding protein (SREBP), stimulatory protein 1 (SP1). SacI and XbaI are the restriction sites of the 175 bp core fragment. Bold arrows indicate 5′ and 3′ ends of the clones from expanding the core SacI/XbaI fragment.

Drug Induction Pattern of 369 bp DRES in LMH Luciferase Reporter Gene Assay

To compare the 369 bp DRES to other inducible fragments, a series of different drugs was used as inducers: clothrimazole (Clo, 10 μM), dexamethasone (Dex, 50 μM), glutethimide (GE, 500 μM), metyrapone (Met, 500 μM), phenobarbital (PB, 500 μM), PCN (10 μM), propylisopropylacetamide (PIA, 250 μM), rifampicin (Rif, 10 μM), RU486 (10 μM), TCPOBOP (10 μM).

FIG. 7 shows induction of the 369 bp DRES by different drugs. Data shown is from two independent experiments. As it was already observed for the 2.8 kb HindIII clone, metyrapone is the strongest inducer, together with glutethimide and PIA.

Mutagenesis of the DR4

All experiments done so far, showed that the region containing the DR4 sites was absolutely required to get any induction at all. To show the direct involvement of the DR4 sites (DR4-1 and DR4-2), they were mutated (in the 369 bp context) in the following way:

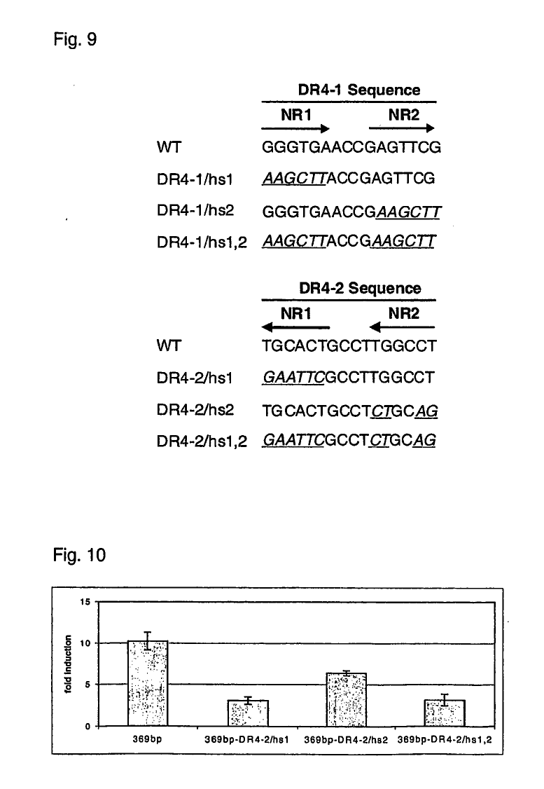

The DR4 halfsites were mutated individually and both together (see FIG. 9). Mutated base pairs are underlined and italic.

To have convenient analysis tools for succesful mutagenesis, the NR1 halfsite was mutated into a Eco-RI site and the NR2 halfsite into a PstI site.

Mutagenesis/Cloning

In a first mutagenesis, each hexamer halfsite was mutated individually. The first PCR was performed using rvp3 plasmid and DR4mt1rv (Seq. Id. No. 33) and glp2 plasmid and DR4mt1fwd (Seq. Id. No. 34) [DR4-2/hs1], DR4mt2rv (Seq. Id. No. 35) and DR4mt2fwd (Seq. Id. No. 36) [DR4-2/hs2] primer pairs, using the 369 bp-LUC clone as template. PCR was run out on 1.5% agarose gel and bands were extracted. The second PCR was performed using the PCR products from the first PCR as template (1μl out of 20 μl for each product) and running a PCR with the external primer rvp3 and glp2. The PCR was run out on a 1.2% agarose gel and the bands were extracted. Then, a KpnI/XhoI digestion was performed on that fragment, and after purification directly ligated to KpnI/XhoI cut pGL3LUCpro +MCS (no gel separation of bands required because after digestion there was only one fragment with the compatible sticky ends for cloning) and heat-shock transformed. Minipreps of DNA were analysed by KpnI/XhoI digestion (insert) and XhoI/EcoRI (DR4-2/hs1, check for mutation) or XhoI/PstI (DR4-2/hs2, check for mutation) digestion. Finally, clones were sequenced to confirm desired mutation and no other bp mutations.

In a second step, both halfsites were mutated. For the double mutant DR4-2/hs1,2, procedure was like above, only that the 369DR4mt2-LUC was taken as template for the first PCR and that the primers DR4mt2mt1fwd (Seq. Id. No. 37) and DR4mt2mt1rv (Seq. Id. No. 38) were used. This construct is shortly termed 369DR4mut-LUC.

Inducibility in LMH Reporter Gene Assay

FIG. 10 shows induction of DR4 mutant constructs of mouse 369 DRES in luciferase reporter gene assay in LMH cells. As inducer metyrapone 500 μM was used. Data shown is from three independent experiments.

Inducibility of the ALA Synthase Drug Responsive Enhancer Sequence by Mouse CAR and Mouse PXR in Transactivation Assays.

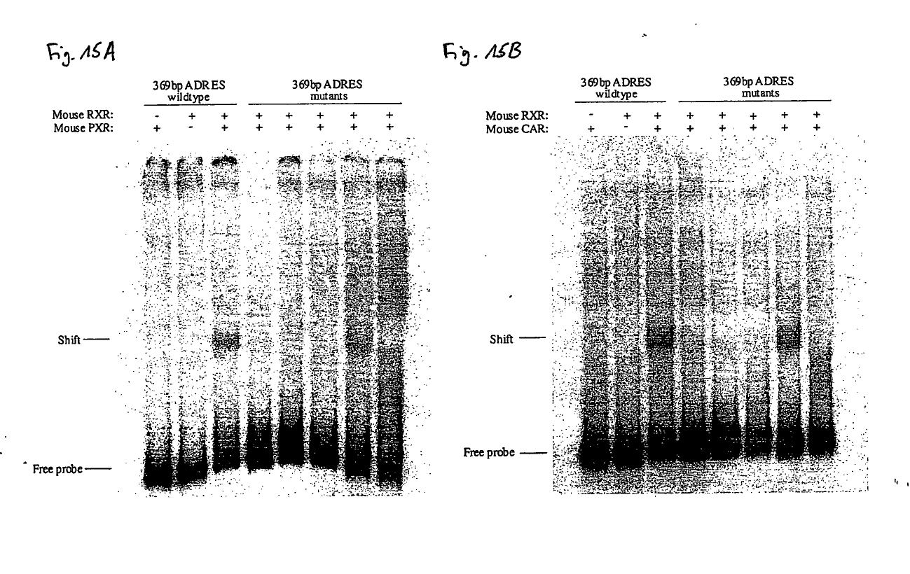

FIG. 14: Transactivations

To test the effects of the mutations on transcriptional activation, transactivations in CV-1 cells with mouse PXR and mouse CAR and their respective inducers PCN and TCPOBOP, were performed (FIG. 14). The inducible DNA constructs were subcloned into the CAT5 reporter vector and tested for their ability to be transactivated by nuclear receptor transcription factors in CV-1 cells. Mouse PXR (A) and mouse CAR (B) coding regions cloned into the pSG5 expression vector were cotransfected along with a vector expressing pSV β-galactosidase as control. Cells were then treated for 16 h with either drugs (A, PCN; B, TCPOBOP) or vehicle control and cell extracts were analyzed for CAT expression normalized against β-galactosidase levels. Experiments were repeated at least three times and error bars represent standard deviations.

As seen in FIG. 14A, the single mutations in the DR4-1 binding site reduced the induction by PCN to 2.7-fold and 2.0-fold, respectively, while the double mutant exhibited 1.9-fold activation. The single mutations in DR4-2 did not significantly reduce induction relative to the wild type value of 5.6-fold, displaying 5.8- and 5.3-fold activation, respectively, whereas the double mutant was reduced to 2.5-fold induction. The alteration of both NR binding sites in the 369 bp element resulted in the elimination of PXR-mediated drug response. In FIG. 14B, CAR transactivations of the DR4-1 single and double mutants did not exhibit any response to TCPOBOP, with even the basal CAR activity eliminated. In comparison, the DR4-2/hs1 and DR4-2/hs2 single mutants still exhibited basal expression that exceeded androstanol-treated levels by 2.5- and 1.9-fold, respectively. TCPOBOP induction exceeded basal levels in the DR4-2/hs1 construct at a modest 1.4 fold, whereas no induction was observed for either the DR4-2/hs2 construct or the double mutant. For the DR4-1,2/hs1,2 quadruple mutant, no basal increase in CAT expression was observed in the absence of androstanol and the induction capacity was completely eliminated.

Both DR4-1 and DR4-2 sites in the 369 bp ADRES element were found to be required for full activation by either PCN or TCPOBOP. Together, the data indicate that DR4-1 is essential for NR-mediated induction via CAR or PXR, whereas DR4-2 might contribute in a more indirect fashion to the overall activation of the 369 bp ADRES. These studies demonstrate an essential contribution of the sequences within the putative DR4 NR binding sites to drug induction of the ADRES elements.

Gel Shift Assays to Determine where Mouse PXR and Mouse CAR Bind within the Enhancer

As both DR4-1 and DR4-2 NR binding sites clearly contribute to the transcriptional activation exhibited by the ADRES elements in transactivations, gel-mobility shift assays were used to determine where PXR and CAR might bind within the enhancer (FIG. 15). Mouse CAR, PXR and RXR proteins were expressed using the TNT T7 Quick Coupled Translation System (Promega) according to the manufacturer's protocol. For DNA fragment labeling, ends were filled in with the Klenow fragment of E. coli DNA polymerase I in the presence of radiolabeled [(α-³²P]ATP and purified over a Biospin 6 chromatography column. A volume of labeled oligonucleotide corresponding to 100,000 cpm was used for each reaction in 10 mM Tris-HCl (8.0)/40 mM KCl/0.05% Nonidet P40/6% glycerol (vol./vol.)/1 mM DTT containing 0.2 μg of poly(dI-dC) and 2.5 μl in-vitro synthesized proteins as described previously (10,12). The reaction mix was incubated for 20 min at room temperature and electrophoresed on a 6% polyacrylamide gel in 0.5×Tris/borate/EDTA buffer followed by autoradiography.

The 369 bp wild type and mutant constructs were examined in the presence of mouse RXR and either mouse PXR (FIG. 15A) or mouse CAR (FIG. 15B). In vitro transcribed/translated mouse RXR or mouse PXR alone bound to the ³²P-radiolabeled 369 bp ADRES (FIG. 15A, lanes 1 and 2), whereas PXR/RXR heterodimers bind the wild type drug responsive enhancer (FIG. 15A, lane 3). PXR/RXR binding to the 369 bp ADRES element was eliminated in the DR4-1/hs1 and DR4-1/hs2 single mutants as well as the DR4-1/hs1,2 double mutant DNA sequences, as demonstrated by the absence of bands in lanes 4, 5 and 6, respectively. Interestingly, the DR4-2/hs1,2 construct still bound the PXR/RXR heterodimer, as seen in lane 7. Moreover, the binding of PXR/RXR heterodimers was absent with the DR4-1,2/hs1,2 quadruple mutant (Lane 8). In a similar fashion, mouse CAR and RXR were unable to bind as homodimers to the 369 bp ADRES, whereas a shift is observed in the presence of RXR/CAR heterodimers (FIG. 15B, lanes 1-3). CAR/RXR binding is also eliminated in the DR4-1/hs1 and DR4-1/hs2 single mutants as well as the DR4-1/hs1,2 double mutant (lanes 4-6, respectively) but still present in the DR4-2/hs1,2 double mutant (lane 7). Not unexpectedly, the DR4-1,2/hs1,2 quadruple mutant does not bind CAR/RXR heterodimers as observed in lane 8. In summary, these findings demonstrate interactions of PXR/RXR and CAR/RXR heterodimers with the DR4-1 but not the DR4-2 binding sites in the 369 bp ADRES elements.

Drug Regulation of the Human Housekeeping ALA-Synthase (ALAS1) Gene

Using a computer-assisted screening approach and sequence information publicly available, several regions of the 5′ flanking region of the ALAS1 were defined as potential mediators of drug-induction of the human housekeeping ALAS gene via nuclear receptors. The defined regions were isolated from a BAC clone containing 30 kb of the upstream region of this gene and introduced into a reporter vector containing the firefly luciferase gene as a reporter for gene activation. These constructs were tested in LMH chicken hepatoma cells, the only known continuously dividing cell line retaining drug-mediated induction.

In these initial screening approaches, two regions were defined which responded to prototypic inducer drugs.

The better-characterized of these regions was called hA795 (Seq. Id. No. 9) and lies approximately 20 kb upstream from the transcriptional start site. By dissecting this fragment, we were able to define a short element, 174 bp (Seq. Id. No. 8) in size, which is sufficient to confer induction in LMH cells. The element was termed hA174. Within this hA174 fragment, a DR4 type nuclear receptor recognition site was found to be necessary for drug induction in site-directed mutagenesis experiments.

The second drug-inducible fragment was called hA8 (Seq. Id. No. 10), is 917 bp in length and is located approximately 16 kb upstream from the transcriptional start site. It contains at least predicted DR-3 and DR-4-type nuclear receptor response elements.

In transactivation assays in a heterologous cell line (monkey kidney CV-1 cells) and human hepatoma HepG2 cells, we assessed which nuclear receptor conveys the observed activation. The two candidate receptors pregnane X receptor (PXR) and constitutive androstane receptor (CAR) were tested on these fragments. With both fragments and the hA174 subfragment, induction by human PXR and activation by mouse and human CAR was observed. The mCAR induced activity could be repressed to approximately 50% by the inverse agonist 3α-androstenol and this repression was reversible by the direct agonist TCPOBOP (1,4-bis[2-(3,5-dichloropyridyloxy)]benzene).

Results

Drug-Induction of Different Fragments of the Human ALAS1 Gene.

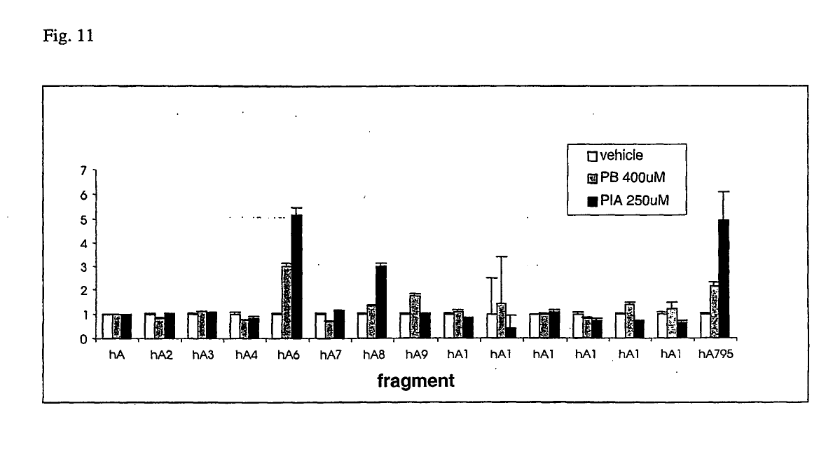

Fragments were cloned into the pGL3 luciferase reporter vector (Promega Corp) and tested for inducibility by the prototypical HALAS1 inducers phenobarbital (PB) and propylisopropylacetamid (PIA). Four hours after transfection of LMH cells with the constructs, cells were exposed to the drugs for 24 hours, after which luciferase activity was assayed. Results were normalized for transfection efficiency by assaying for activity of co-transfected β-galactosidase. Data shown is one representative experiment (FIG. 11).

The Effect of Drugs on the hA795 Element Depends on the Presence of a DR-4 Motif.

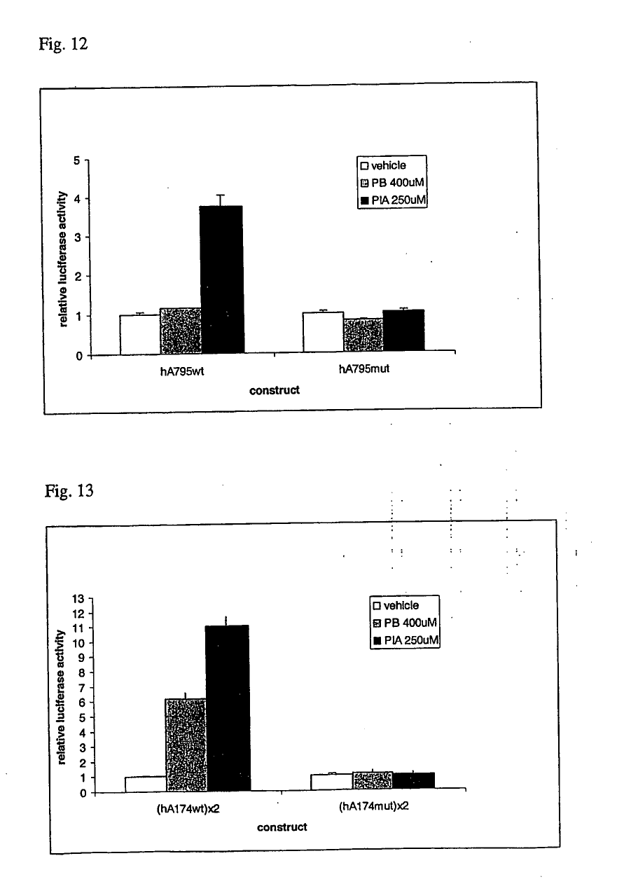

Within the sequence of the hA795 fragment (Seq. Id. No. 9), a putative DR-4 type nuclear receptor response element was found by computer analysis. Site-directed mutagenesis of this element abolished inducibility of this fragment in reporter gene assays in LMH cells. Experiments were performed as described above (FIG. 12).

A Core Sequence Spanning the DR-4 Element is Sufficient to Mediate Drug Induction in LMH Cells.

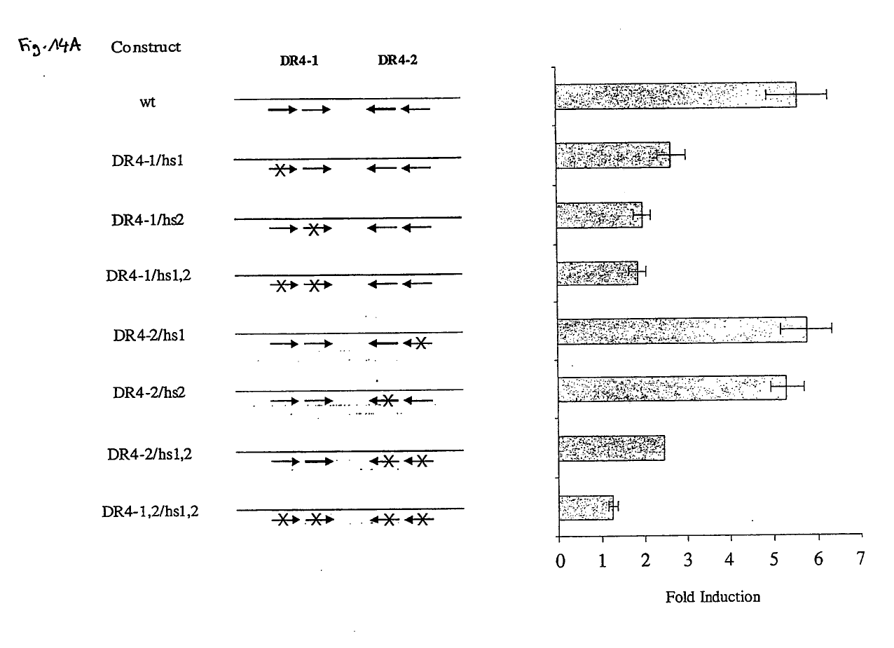

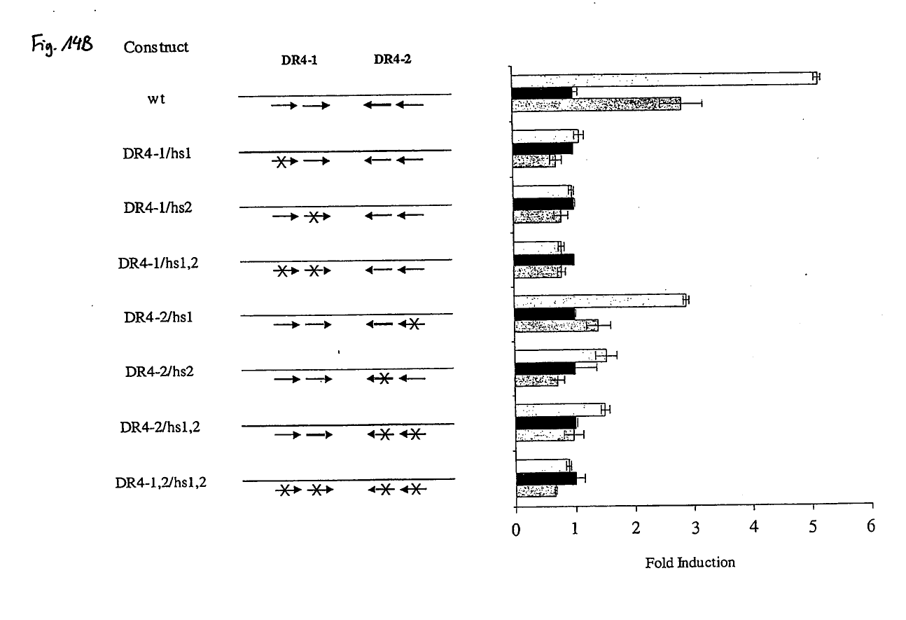

From the hA795 fragment, the hA174 fragment was derived (Seq. Id. No. 8). It is 174 bp in length and within its sequence, the DR-4 response element is contained. Direct repeats of the wildtype hA174 or a mutant, where the DR-4 was destroyed were cloned into the pGL3 reporter vector and tested in LMH cells (FIG. 13). Data is from one representative experiment.

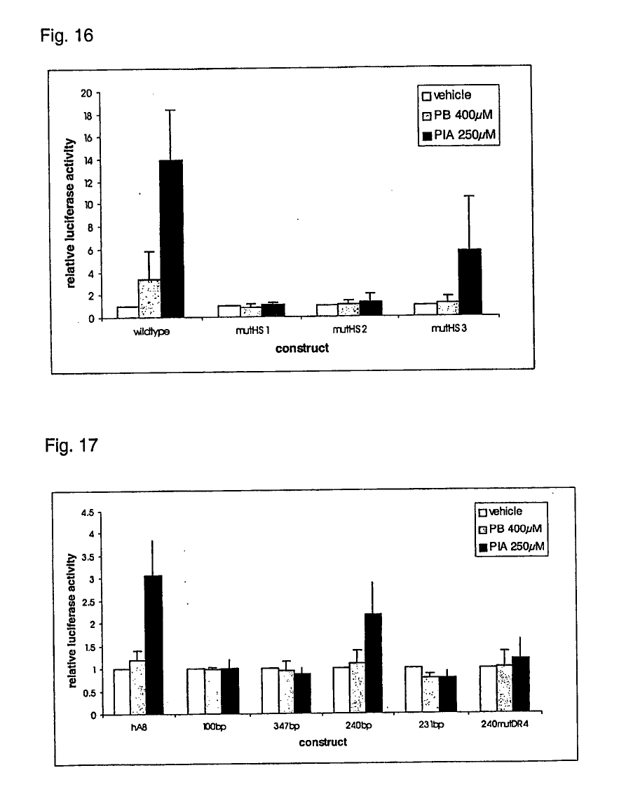

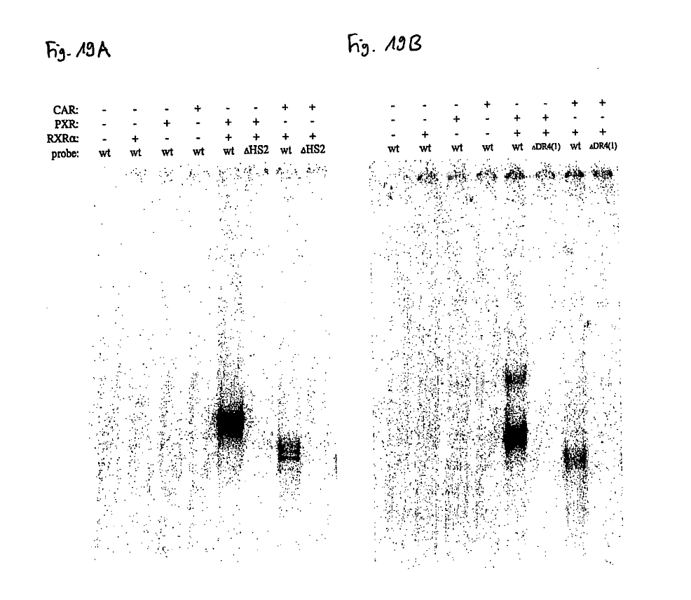

FIG. 16 shows the hA174 core element derived from the hA795 fragment conferring drug-mediated transcriptional activation to a reporter gene in LMH cells. We investigated in more detail the contribution of various response element half-sites to drug-induction mediated by this element. Within the hA174 element, an arrangement of three nuclear receptor response element half-sites was found, which lead: to the prediction of a DR-4 type, as well as a DR-5 type response element (of. FIG. 18A). By site-directed mutagenesis, we examined the involvement of each of these half-sites to drug induction. In the context of the 2xhA174 reporter construct, each single half-site was mutated in both copies of the hA174 element. The wild-type and mutant constructs were subsequently tested for reporter gene activation in LMH cells as described above. Cells were induced with 400 μM phenobarbital (PB) or 250 μM propylisopropylacetamid (PIA). As a control, vehicle alone (0.1% DMSO) was added to the cells. After 24 hours, luciferase activity was assayed and normalized against co-transfected β-galactosidase activity in order to compensate for transfection efficiency. Mutagenesis of the HS1 or HS2 half-site leads to abolishment of inducibility of this fragment, whereas alteration of the HS3 half-site merely reduces induction levels. From this, we conclude that the DR-4 element, consisting of the HS1 and HS2 half-sites, is essential for drug induction. The change in induction observed by mutagenesis of the HS3 half-site may indicate a modulatory role for the half-site alone or as a DR-5 in combination with HS2 in drug induction. Data shown are the mean+1SD of three independent experiments.

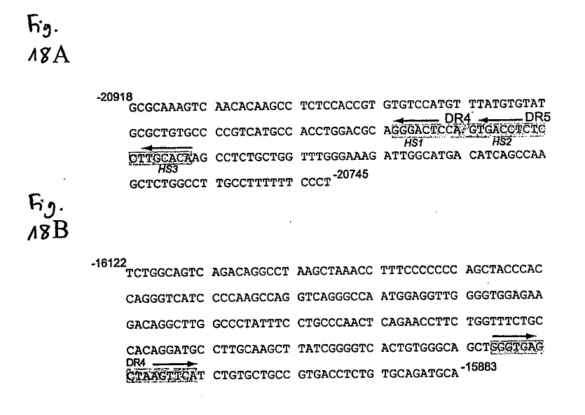

FIG. 17 shows the sub-fragmentation of the hA8 drug-responsive element (Seq. Id. No. 10).

In order to define a core sequence retaining inducibility, the hA8 fragment (Seq. Id. No. 10), which carries PCR-introduced KpnI and XhoI restriction sites at its ends, was digested with SacI and NsiI. The resulting four fragments, a 100 bp KpnI/SacI fragment, a 374 bp SacI/NsiI fragment, a 240 bp NsiI/NsiI fragment, and a 231 bp NsiI/XhoI fragment, were cloned into a reporter gene vector and subsequently tested for drug induction in LMH cells. Of these fragments, only the 240 bp fragment (called hA240) (Seq. Id. No. 39) was able to convey transcriptional activation in response to drugs. Within the core hA240 fragment, we then mutated both half-sites of a predicted DR-4 element (cf. FIG. 18B) and observed the effect of this mutation on drug induction (240mutDR4). Alteration of the DR4 element half-sites abolished drug induction in LMH cell reporter gene assays. Data shown are the mean+1SD of three independent experiments.

FIG. 18 depicts in detail the core elements of the two drug-responsive regions within the human ALAS1 gene. The position numbers refer to the position of these sequences in the human genome, relative to the transcriptional start site of the human ALAS1 transcript. Response elements are boxed in grey, and individual half-sites are marked with arrows above and, if necessary for distinction, labels below the sequence. In A, the hA174 bp core (Seq. Id. No. 8) is depicted. The grey box indicates the DR-4/DR-5 site cluster, made up of three individual half-sites. In B, the 240 bp core is shown (Seq. Id. No. 39), with a grey box and arrows marking the functional DR-4 element.

FIG. 19 depicts the results of an electrophoretic mobility shift assay, assaying the ability of human PXR and human CAR to bind to the hA174 and hA240 elements. Binding of these nuclear receptors happens in a heterodimeric state with the human 9-cis retinoic acid receptor RXRα. From expression plasmids encoding human RXRα, RXR or CAR, receptor protein was made in vitro using the TNT reticulocyte lysate coupled transcription/translation system (Promega Corp.).

FIG. 19A shows an assay using radiolabeled hA174 wildtype fragment or hA174 fragment, where half-site 2 was mutated (cf. FIG. 18A). Lanes 1-4: Wildtype hA174 was incubated with no receptor (mock transcription/translation) or with RXRα, PXR or CAR alone. No complex is observed using single receptors. Lanes 5 and 7: Wild-type hA174 was incubated with RXRα and PXR or RXRα and CAR, upon which complex formation between radiolabeled probe and protein is observed. Lanes 6 and 8: Same conditions as in lanes 5 and 7, but instead of wild-type hA174, a radiolabeled probe was used where the half-site 2 was mutated. No complex is formed anymore.

FIG. 19B: Instead of the hA174 fragment, wild-type or DR4-mutant hA240 fragment was radiolabeled and used as probe. Lanes 1-4: Wildtype hA240 was incubated with no receptor (mock transcription/translation) or with RXRα, PXR or CAR alone. No complex is observed using single receptors. Lanes 5 and 7: Wild-type hA240 was incubated with RXRα and PXR or RXRα and CAR, upon which complex formation between radiolabeled probe and protein is observed. Lanes 6 and 8: Same conditions as in lanes 5 and 7, but instead of wild-type probe, radiolabeled hA240DR4(2) mutant was used. No complex is formed.

The abbreviations used are: ALAS, 5-aminolevulinic acid synthase; ADRES, aminolevulinic acid drug responsive enhancer sequence; PB, phenobarbital; DR, hexamer half-site direct repeat; h, hours; bp, basepairs; LMH, leghorn male hepatoma; kb, kilobases; NF1, nuclear factor 1; CYP, cytochrome(s) P450; CXR, chicken xenobiotic receptor; PXR, pregnane X receptor; CAR, constitutive androstane receptor; RXR, 9-cis-retinoic acid receptor; PIA, propylisopropylacetamide; PCN, 5-pregnen-3β-ol-20-one-16α-carbonitrile; TCPOBOP, 1,4-bis[2-(3,5dichloropyridyl-oxy)]benzene; LUC, luciferase; mifepristone, RU-486; clotrimazole, 1-[o-chlorotrityl]-imidaszole; EMSA, electrophoretic mobility shift assay; cpm, counts per minute; FCS, fetal calf serum.

While there are shown and described presently preferred embodiments of the invention, it is to be distinctly understood that the invention is not limited thereto but may be otherwise variously embodied and practiced within the scope of the following claims.

### REFERENCE

1. Boshart, M., M. Kluppel, A. Schmidt, G. Schutz, and B. Luckow. 1992. Reporter constructs with low background activity utilizing the cat gene. Gene 110:129-30.
2. Handschin, C., M. Podvinec, and U. A. Meyer. 2000. CXR, a chicken xenobiotic-sensing orphan nuclear receptor, is related to both mammalian pregnane X receptor (PXR) and constitutive androstane receptor (CAR). Proc Natl Acad Sci USA 97:10769-74.
3. Honkakoski, P., I. Zelko, T. Sueyoshi, and M. Negishi. 1998. The nuclear orphan receptor CAR-retinoid X receptor heterodimer activates the phenobarbital-responsive enhancer module of the CYP2B gene. Mol Cell Biol 18:5652-8.
4. Iniguez-Lluhi, J. A., D. Y. Lou, and K. R. Yamamoto. 1997. Three amino acid substitutions selectively disrupt the activation but not the repression function of the glucocorticoid receptor N terminus. J Biol Chem 272:4149-56.
5. Kim, J., G. Min, and B. Kemper. 2001. Chromatin assembly enhances binding to the CYP2B1 Phenobarbital-responsive unit (PBRU) of nuclear factor 1, which binds simultaneously with constitutive androstane receptor (CAR)/retinoid X receptor (RXR) and enhances CAR/RXR-mediated activation of the PBRU. J Biol Chem 276:7559-7567.
6. Kliewer, S. A., J. M. Lehmann, and T. M. Willson. 1999. Orphan nuclear receptors: shifting endocrinology into reverse. Science 284:757-60.
7. Kliewer, S. A., J. T. Moore, L. Wade, J. L. Staudinger, M. A. Watson, S. A. Jones, D. D. McKee, B. B. Oliver, T. M. Willson, R. H. Zetterstrom, T. Perlmann, and J. M. Lehmann. 1998. An orphan nuclear receptor activated by pregnanes defines a novel steroid signaling pathway. Cell 92:73-82.
8. Lehmann, J. M., D. D. McKee, M. A. Watson, T. M. Willson, J. T. Moore, and S. A. Kliewer. 1998. The human orphan nuclear receptor PXR is activated by compounds that regulate CYP3A4 gene expression and cause drug interactions. J Clin Invest 102:1016-23.
9. Rupp, R. A., U. Kruse, G. Multhaup, U. Gobel, K. Beyreuther, and A. E. Sippel. 1990. Chicken NFI/TGGCA proteins are encoded by at least three independent genes: NFI-A, NFI-B and NFI—C with homologues in mammalian genomes. Nucleic Acids Res 18:2607-16.
10. Savas, U., K. J. Griffin, and E. F. Johnson. 1999. Molecular mechanisms of cytochrome P-450 induction by xenobiotics: An expanded role for nuclear hormone receptors. Mol Pharmacol 56:851-7.
11. Waxman, D. J. 1999. P450 gene induction by structurally diverse xenochemicals: central role of nuclear receptors CAR, PXR, and PPAR. Arch Biochem Bio- phys 369:11-23.

1. An isolated nucleic acid sequence comprising at least a DR-4 nuclear receptor binding sites wherein said nucleic acid sequence functions as trancriptional enhancer of the 5-aminolevulinic acid synthase gene.

2. The nucleic acid sequence of claim 1 with the proviso that said sequence does not comprise a sequence set forth in Seq. Id. No. 8 to 10.

3. The nucleic acid sequence of claim 1, wherein said sequence comprises the sequence set forth in Seq. Id. No. 1.

4. The nucleic acid sequence of claim 1, wherein said nucleic acid sequence further comprises a nuclear factor 1 binding site (NF-1) and/or a DR-5 nuclear receptor binding site.

5. The nucleic acid sequence of claim 1, wherein said nucleic acid sequence mediates chemical compound induced transcriptional activation.

6. The nucleic acid sequence of claim 5, wherein said chemical compound is a candidate compound for therapeutical use or a drug.

7. The nucleic acid sequence of claim 1, wherein said sequence comprises a sequence selected from the group consisting of Seq. Id. Nos. 2 to 6 and Seq. Id. No. 7.

8. A genetic construct comprising a nucleic acid sequence of claim 1, wherein said nucleic acid is operably linked to a nucleic acid encoding a reporter molecule.

9. The genetic construct of claim 8, wherein said reporter molecule has an enzymatic activity.

10. The genetic construct of claim 9, wherein said reporter molecule activity can be detected by colorimetry, radioactivity, fluorescence or chemiluminiscence.

11. The genetic construct of claim 8, wherein said reporter molecule is selected from the group consisting of luciferase, beta-galactosidase, chloramphenicol acetyltransferase, alkaline phosphatase and green fluorescent protein.

12. A method for testing compounds for modulation of heme and/or P 450 cytochromes synthesis comprising contacting suitable cells comprising a genetic construct according to claim 8 with a test compound and detecting enhanced or repressed expression and/or transcription of the nucleic acid sequence encoding the reporter gene.

13. The method of claim 12, wherein said compound is a candidate drug for therapeutical use or a drug.

14. The method of claim 12, wherein enhanced expression of the nucleic acid sequence encoding the reporter gene is detected by a colorimetry, fluorescence, radioactivity or chemiluminiscence.

15. The method claim 12, wherein enhanced transcription of the nucleic acid encoding the reporter gene is detected by quantitative PCR.

16. The method of claim 12, wherein said cells are Leghorn Male Hepatoma (LMH) cells, other hepatoma cells, monkey kidney cells (CV-1, COS-1) or human kidney cells.

17. (canceled)

18. (canceled)

19. An isolated nucleic acid sequence comprising at least a DR-4 nuclear receptor binding site, wherein said nucleic acid sequence functions as transcriptional enhancer upon addition of a compound that induces a 5-aminolevulinic acid synthase gene.

20. A method for testing at least one testing compound as a modulator of heme and/or P450 cytochromes synthesis comprising:
providing an expression system comprising the isolated nucleic acid sequence of claim 19,
adding said at least one testing compound, and
ascertaining modulation of expression levels in said expression system, wherein said modulation is mediated by said nucleic acid sequence.

21. The method of claim 20, wherein said isolated nucleic acid sequence is Seq. Id. No. 8, Seq. Id. No. 9, Seq. Id. No. 10 or Seq. Id. No. 39.

22. The method of claim 20, wherein said isolated nucleic acid is operably linked to a nucleic acid encoding a reporter molecule.
